(19) United States
(12) Patent Application Publication
(10) Pub. No.: US 2006/0127425 A1
(43) Pub. Date: Jun. 15, 2006

(54) Encapsulated material released to generate perceivable sensorial indicia of discrete event occurrence

(75) Inventors: John E Walls, Longmont, CO (US); Jeffrey W Putt, Fort Collins, CO (US); Kenneth E Deline, Avon, CO (US)

Correspondence Address: CR MILES, P.C., 1 OLD TOWN SQUARE, SUITE 200 B, FORT COLLINS, CO, 80524, US

(73) Assignee: Venture Management Alliance, LLC, Greeley, CO (US)

(21) Appl. No.: 10/540,716

(22) PCT Filed: Dec. 10, 2003
(86) PCT No.: PCT/US03/39472

§ 371 Date: Jun. 10, 2005

Related U.S. Application Data
(60) Provisional application No. 60/432,471, filed on Dec. 10, 2002.

(51) Int. Cl. A61K 8/02

(52) U.S. Cl. 424/401

(57) ABSTRACT

Compositions (1) that provide a carrier (2) to covey a plurality of capsules (3) containing materials) (4) released to generate perceivable sensorial indicia discrete event occurrence and which can further provide perceivable sensorial reinforcers as incentives or disincentives to composition use coincident with such discrete events.

This International Patent Cooperation Treaty Application claims the benefit of U.S. Application No. 60/432,471, filed Dec. 10, 2002, hereby incorporated by reference herein.

### I. TECHNICAL FIELD

Compositions that provide a carrier to covey capsules containing material(s) released to generate perceivable sensorial indicia discrete event occurrence and which can further provide perceivable sensorial reinforcers as incentives or disincentives to composition use coincident with such discrete events.

Specifically, a hand washing system that provides capsules containing material to generate perceivable sensorial indicia of a desired level, duration, or efficacy of hand washing and which can further provide one or more perceivable sensorial reinforcers to encourage attainment of the desired level, duration, or efficacy of hand washing.

### II. BACKGROUND

Compositions, such as cosmetics and hand washing agents, typically comprise a carrier system which can contain conventional perceivable sensorial attributes. A conventional sensorial attribute can be a fragrance. Pleasing odor can induce persons to purchase and re-purchase a particular cosmetic product. The manner in which the fragrance is released can also be an important factor influencing a person's physiology and psychology as described by U.S. Pat. No. 5,238,915.

Another conventional sensorial attribute can be color. Colorants can be added to cosmetic products to render them consistent in color or can be added to cosmetic products in a manner to create decorative patterns or to differentiate discrete portions of the cosmetic product as described in U.S. Pat. No. 6,147,040, or International Patent Application No. WO 99/40172. Certain cosmetic products are provided with an initial colorant or a secondary colorant within capsules which generate color or color changes of the initial colorant upon rupture of the capsules as described by U.S. Pat. Nos. 4,756,906 and 5,320,835.

In certain compositions the sensorial attribute is that of temperature. Coolness can be imparted to cosmetic products such as toothpastes or aftershave lotions through the presence of xylitol, camphor, menthol, or menthol derivatives such as menthol lactate as described by International Patent Application WO 01/12148, or U.S. Pat. No. 5,861,440.

Other cosmetic products can provide an exothermic temperature increase by inclusion of capsaicin or aluminosilicate interacting with water, each providing a brief exothermic reaction as a source of heat as described by U.S. Pat. Nos. 4,379,143; 4,626,550; 4,362,715; or 3,250,680. Certain cosmetic products even provide a two-stage temperature rise, such as, the two-stage chemically heated soap composition described by U.S. Pat. No. 4,839,081. The temperature rise may even be accompanied by exothermic effervescence which may be appealing in itself or may help disperse other perceivable sensorial attributes, such as fragrance, as described by U.S. Pat. No. 5,993,854.

Feel of a product can be highly important. Creams, lotions, gels or pastes can be adjusted to provide the desired tactility. A graded series of tactility from silly to tacky can be provided as disclosed by International Patent Application WO 01/12148.

While each these perceived sensorial attributes may be added to compositions, formulations, or cosmetics to address the concerns of personal taste they do not generate indicia of discrete event occurrence, such as an indicator of therapeutic efficacy or an indicator of compliance with an established guideline for product use, or the like, nor do they provide perceivable sensorial reinforcers to encourage or discourage coincidence of composition or cosmetic use with the occurrence of a discrete event(s).

As but one non-limiting example, conventional hand washing agents or compositions typically include one or more conventional perceived sensorial attributes such as color, fragrance, or tactility, but none of these perceived sensorial attributes provide any indicia as to the efficacy of the particular hand washing event, or indicia as to when compliance with hand washing procedure(s) or guidelines have been met when using a particular hand washing agent, or other composition. Additionally, none of these conventional perceived sensorial attributes are coupled or coordinated with particular hand washing events in a manner that increases the probability that the user will be compliant with hand washing procedures or guidelines for that hand washing agent.

As such, while conventional hand washing procedures utilizing soap and water may be the single most effective practice that prevents the spread of germs and while experts unequivocally agree that conventional hand washing with soap and water can reduce the incidence of nosocomial infections, compliance with hand washing guidelines is embarrassingly and unacceptably low. United States Department of Health and Human Services, Center For Disease Control, “Hospitals Abandon Soap, Water; Alcohol Rinses Kill Germs Better” (2002). In fact, studies consistently demonstrate that rates of hand washing compliance is less than 50%. “A Survey On Handwashing Practices And Opinions Of Healthcare Workers”, Journal of Hospital Infection, 45(4): 318-321 (2000). This failure to comply with hand washing guidelines may contribute to nosocomial infections which have been shown to complicate between six and fourteen percent of admissions in pediatric institutions. Posfay-Barbe, Kiara and Pittet, Didier, “New Concepts In Hand Hygiene”, W.B. Saunders Company, (2001). These hospital infections are the fourth leading cause of death. Many of the deaths are caused by unsanitary facilities, germ laden instruments, and unwashed hands. “Hospital Infections Fourth Leading Cause of Death”, The Associated Press, The Coloradoan, Jul. 21, 2002, p. A3.

Similarly, many millions of non-health care workers get sick with colds, flu or stomach viruses that could be prevented by proper hand washing. The Center for Disease Control (CDC) estimates that nearly 22 million school days are lost each year due to colds alone, and that 75% of those colds result in visits to the doctor's office. In addition, the CDC reports that, out of the 95 million cases of influenza reported annually, 70-million work-loss days result. The CDC states that hand washing is the single most effective practice that prevents the spread of germs.

Because of the prevalence of non-compliance with hand washing guidelines and the size of the commercial market in which hand washing products can be sold, a variety of conventional technologies have been proposed to improve hand washing compliance. These include devices used when hands are washed, such as, automated sinks with water flow and soap dispensing controlled by electronic sensors as described by Larson, E., McGeer A., Quarisi, A., et al., “Effect of an automated sink on handwashing practices and attitudes in high-risk units”, Infec. Control Hosp. Epdeniol. 12:442-448 (1991); and can further include hand washing machines as described by Decker, L., Gross, A., Miller, Read, J., Cutright, D. and Devine, J., “A Rapid Method For The Presurgical Cleansing Of Hands”, Obstet. Gynecol. 51:115-117 (1978). Certain conventional technologies also include soap dispensers with timed signaling activated at the time soap is dispensed which signals the user after a predetermined interval indicating that the washing time is over as disclosed by U.S. Pat. No. 5,771,925.

Even though numerous conventional technologies have been developed to promote the proper application and use of compositions, cosmetics, and hand washing agents in general, and specifically to meet procedural guidelines or regulations with respect to hand washing, a number of problems remain unresolved with the proper application and use of conventional composition, cosmetic, and hand washing technologies (hereinafter encompassed by the term “conventional cosmetic technologies”).

A significant problem with conventional cosmetic technologies can be that while the cosmetic user understands the importance of proper application and use of a cosmetic agent, they tend to overestimate their own compliance with procedures or guidelines for the cosmetic. With respect to hand washing, as an example, healthcare workers understand the importance of hand washing, but they tend to overestimate their own compliance. “Effectiveness Of A Hospital-Wide Program To Improve Compliance With Hand Hygiene”, Lancet, 356 (9238): 1307-1312 (2000). Thus, even when various conventional technologies are made available they may not be used because users may incorrectly believe they are all ready using the cosmetic properly, or are in compliance with guidelines, such as hand washing guidelines.

Another significant problem with conventional cosmetic technologies can be cosmetic user distaste for external intervention. For example, healthcare workers are not in favor of interventions involving rewards and punishments to achieve hand washing compliance. Pittet, D., “Effectiveness Of A Hospital-Wide Program To Improve Compliance With Hand Hygiene”, Lancet, 356 (9238): 1307-1312 (2000). As such, technologies which provide portable, individualized, hand washing agent dispensers which track and store into memory the site where workers dispense hand wash agents as described by U.S. Pat. No. 6,392,546; or the use of modules at wash sinks into which employees input identification codes to track compliance, may actually reduce compliance or may be disfavored by employees.

Another significant problem with conventional cosmetic technology may be that ineffective or less effective conventional cosmetic technologies are selected when they are easier to use. Healthcare workers are attracted to interventions that make handwashing easier. Pittet, D., “Effectiveness Of A Hospital-Wide Program To Improve Compliance With Hand Hygiene”, Lancet, 356 (9238): 1307-1312 (2000). As such, heath care workers may become reliant on premoistened towelettes, waterless hand cleaners, or wipes. United States Department of Health and Human Services, Center For Disease Control, The ABC's of Safe and Healthy Child Care (2002). However, pre-moistened towelettes do not effectively clean and do not take the place of hand washing. Waterless hand cleaners or pre-moistened towelettes should not be used as a substitute for washing hands with soap and water. Towelettes should only be used to remove residue, such as food off a baby's face or feces from a baby's bottom during diaper changing.

Another significant problem with conventional cosmetic technology may be that alternative cosmetics used for the same or similar procedure may have different performance characteristics. For example, various types of antiseptic ingredients included in cosmetic compositions, may have different performance characteristics. A first characteristic of antiseptic agents that can differ is time to achieve maximum reduction in bacteria counts. Alcohol based preparations, for example, require less time to effect maximum reduction in bacterial counts than do products containing chlorohexidine gluconate (CHG). Indeed, a one-minute immersion or scrub with alcohol is as effective as a four to seven minute skin preparation with other antiseptics. Hexacholrophene on the other hand may not be particularly fast acting and its rate of killing is slow to intermediate. Larson, Elaine, “APIC Guideline For Hand Washing And Hand Antisepsis in Health-Care Settings” (1995). A second characteristic of antiseptic agents that can differ is their ability to bind to the stratum corneum, resulting in a persistent activity on the skin. For example, CHG may be affected by individual differences in skin pH, secretions, or moisture level. A third characteristic of antiseptic agents which may differ can be concentration to achieve microbiocidal activity. For example, free iodine is the major chemical and microbiocidal factor in the activity of iodophors and changes with the degree of dilution. Id.

Perhaps common to each of these prominent problems with conventional cosmetic technology may be the lack of any perceivable sensorial indicia generated by the composition, cosmetic, or hand washing agent to inform the user of occurrence of discrete event(s), such as, achievement of a standard of compliance with procedures utilizing a particular cosmetic, or attainment of therapeutic efficacy with the cosmetic. As such, provision of feed back to the user of conventional cosmetics involves additional mechanical timing or signaling devices, activity recording or retrieval devices, or the like, some of which are described above in the context of hand washing compliance.

Additionally, conventional cosmetic technology may lack any perceivable sensorial reinforcement to increase the probability, or decrease the probability, of achieving or attaining the above described discrete event occurrence. For example, health care workers can be induced to perform hand washing from the use of strategically placed reminders, or asking patients to remind staff of the need to conduct hand washing, each having a positive effect on compliance. Naikoba, S. and Hayward, A., “The Effectiveness Of Interventions Aimed At Increasing Handwashing In Healthcare Workers—A Systematic Review”, The Hospital Infection Society 0195-6701/01/030173+08. However, these types of reinforcement require the intervention of other parties which may not always be available, reliable, or consistent.

A problem prominent within the broad context of conventional cosmetic compositions, and specifically with regard to providing perceivable sensorial indicia or perceivable sensorial reinforcers, as described in the non-limiting examples provided above, may be that a cosmetic carrier cannot contain, or act as the conveyance for, components, elements, or compositions which provide the perceivable sensorial indicia in a manner that can be coupled to discrete event occurrence. One aspect of this problem may be that the cosmetic carrier itself may contain water sufficient to activate such components, elements, or compositions used as perceivable sensorial indicia of discrete event occurrence.

As such, aqueous activated perceivable sensorial indicia of discrete event occurrence may not reside in or be conveyed by the carrier for the desired length of time without being activated by the carrier itself. A second aspect of this problem can be that even though the carrier does not contain water sufficient to activate aqueous activated perceivable sensorial indicia of discrete event occurrence, the carrier is not water miscible. This may preclude the carrier from being used in applications in which the carrier and water must readily form a single phase. A third aspect of this problem may be that the carrier may interfere with the activation, dissemination, or attributes of the aqueous activated components conveyed. A fourth aspect of this problem may be that while the carrier is suitable to convey perceivable sensorial indicia of discrete event occurrence, the carrier(s) is/are not suitable for application to the skin.

The present invention provides compositions, cosmetics, and hand washing agents which address each of the above-mentioned problems.

### III. DISCLOSURE OF INVENTION

Accordingly, a broad object of the invention can be to provide capsules containing material(s) that remain intact in a composition or carrier until delivery of predetermined application characteristics to the composition (such as an amount or level of pressure, shear, or the like, individually or in combination, whether delivered substantially instantly or delivered over a duration of time) or particular process step characteristics commence, or an elapse of a specific duration of time with proper utilization of the composition, or the like, and thereafter releases material to generate sensorial attributes.

Another broad object of the invention can be to provide capsules containing material(s) that remain intact in a composition or carrier until delivery of predetermined application characteristics to the composition (such as an amount or level of force, pressure, shear, individually or in combination) or particular process step characteristics commence, or an elapse of a specific duration of time of proper utilization of the composition, or the like, and then release material to generate sensorial indicia of occurrence of a discrete event to the user.

A non-limiting example of providing sensorial indicia of discrete event occurrence pertains to certain hand washing agents in which the concentration of iodophore present at the beginning of hand washing procedure may not be or have less than desired efficacy. By providing indicia of the discrete event occurrence of an efficacious iodophore concentration, the hand washer can be informed or understand when the level of iodophore present during a hand washing procedure is sufficient to reduce bacterial populations.

Providing sensorial indicia of discrete event occurrence can be particularly useful when the composition has a utility or application that does not otherwise provide perceivable sensorial indicia of efficacy or performance to the user, such a therapeutic efficacy; or when the cosmetic application requires performance of a plurality of different steps, or when the cosmetic requires serial timed application, or when one or more cosmetic agents need to be mixed to achieve a desired result, or the like.

Another broad object of the invention can be to provide capsules that release material to generate perceivable sensorial reinforcers in response to predetermined application characteristics that increase or decrease the probability of coincidence of composition use with occurrence of a discrete event. The various approaches to utilizing the invention to achieve this object can be consistent with the traditional reinforcement schedules or operant conditioning theories, if desired, to increase the probability of attaining a discrete event or to decrease the probability of a attaining a discrete event. As such, one aspect of the invention can be to provide one or more perceivable sensorial reinforcers that are operably coupled to application characteristics so that material release from the capsule generates and incentive or reward with respect to composition at a time coincident with the discrete event. Utilizing generated perceived sensorial reinforcers as conduit to couple composition use to a discrete event occurrence in this manner differentiates it from perceived sensorial indicia which only provides notice that a discrete event has occurred.

For example, a hair conditioner composition in accordance with the invention may requires an amount of mechanical or physical manipulation with hair to achieve a desired degree of efficacy can include capsules that release material to generate a perceived sensorial indicia such color to indicate that sufficient manipulation of the hair conditioner composition with the hair achieved. The hair conditioner composition can further include capsules that release materials to generate perceived sensorial reinforcer(s) to provide incentives between commencement of hair conditioner use and achievement of sufficient mechanical or physical manipulation. The perceived sensorial reinforcer could be a fragrance released after commencing use of the hair conditioner composition and before release of material from capsules to generate the perceived sensorial indicia of color change to indicate sufficient manipulation of the hair conditioner composition with the hair, thereby inducing sufficient manipulation of the hair conditioner composition with the hair.

Another example of a hair conditioner composition in accordance with the invention can comprise a combined perceived sensorial reinforcer with a perceived sensorial indicia through use of capsules that rupture to generate increasing color intensity until the discrete event occurs.

Another specific object of the invention can be to provide hand washing agents having a capsule that releases material to generate perceivable sensorial attributes (differentiated from perceivable sensorial indicia because release of material to generate the attribute is uncoupled from or not coordinated with discrete event occurrence) after hand washing has begun selected from the group consisting of at least one perceivable colored material not the perceivable color of the hand washing agent prior to hand washing; a first perceivable color and a second perceivable color neither of which are the perceivable color of the hand washing agent prior to hand washing; a first perceivable color, a second perceivable color, and a third perceivable color none of which are the perceivable color of the hand washing agent prior to hand washing; an audible sound have a decibel level greater than typically generated by use of the hand washing agent itself; a first fragrance not the fragrance of the hand washing agent perceived prior to hand washing; a first and a second fragrance neither of which are the fragrance perceived prior to hand washing; or a tactility perceivable only after hand washing has begun, or the like.

Another specific object of the invention can be to provide hand washing agents having capsules that release perceivable sensorial indicia of discrete event occurrence(s), such as completion of a desired hand washing duration, or attainment of a level of hand washing having a desired degree of therapeutically efficacy, or the like. As to these embodiments of the invention, the perceivable sensorial indicia of discrete event occurrence(s) can be selected from the group of: at least one color not perceivable until the discrete event occurs; color intensity not perceivable until the discrete event occurs; a first perceivable color and a second perceivable color neither of which are perceivable until the discrete event occurs; a first perceivable color, a second perceivable color, a third perceivable color, none of which are the perceivable until the discrete event occurs; a first color and a second color, the first color perceivable after hand washing beings and the second color not perceivable until the discrete event occurs; a first color, a second color, and a third color, the first and second color perceivable serially after hand washing begins, the third not perceivable until the discrete event occurs; a color not perceivable until the discrete event occurs and then perceivable for a duration of time and then becoming unperceivable; an audible sound having a decibel level greater than typically generated by use of hand washing agents not perceivable until the discrete event occurs; a first audible sound, and a second audible sound having perceivably greater decibel value than the first audible sound, the first audible sound perceivable after hand washing has begun, the second audible sound not perceived until the discrete event occurrence; a first fragrance not perceived until the discrete event occurrence; a first and a second fragrance, the first perceived during hand washing, the second not perceived until discrete event occurrence; or a tactility not perceivable until discrete event occurrence, including the various combinations and permutations thereof.

Another specific object of the invention can be to provide hand washing agents having capsule(s), of a single type or multiple types, that release(s) perceivable sensorial reinforcer(s) to adjust the probability of achieving a desired hand washing duration, or attaining desired amount or level of hand washing corresponding to therapeutically efficacy. As to these embodiments of the invention, the perceivable sensorial reinforcers to adjust the probability of discrete event occurrence can be selected, for example, from the group of: a color perceivable at the time hand washing begins increasing in intensity at least until perceivable sensorial indicia of discrete event occurrence; a color not perceivable during hand washing until the perceived sensorial indicia of discrete event occurrence and then lasting for a duration of time substantially exceeding the indicia period, wherein the indicia period could have a duration of time between about 1 second to about 5 seconds (although the duration from embodiment to embodiment of the invention may vary); at least one color perceivable after hand washing begins at least until the perceivable indicia of discrete event occurrence; a fragrance perceivable after hand washing begins but prior to discrete event occurrence.

These and other objects of the present invention will become more readily apparent from consideration of the following summary and detailed discussion.

### IV. BRIEF DESCRIPTION OF DRAWINGS

FIG. 1 illustrates a composition in accordance with the invention which provides a carrier conveying capsules having adjustable capsule rupture characteristics containing materials which release to generate perceivable sensorial attributes, perceivable sensorial indicia of discrete event occurrence, or sensorial reinforcers.

FIG. 2 provides a graph which shows the rate of release of material from capsules in accordance with the invention which generates a perceived sensorial indicia (color generation and sound generation indicating elapse of a duration of time consistent with therapeutic use of the composition) of a discrete event occurrence.

FIG. 3 provides a graph which shows the rate of release of material from a capsule in accordance with the invention which generates a perceived sensorial indicia (heat generation and fragrance generation) of a discrete event occurrence.

FIG. 4 provides a graph which shows the rate of release of material from a capsule in accordance with the invention which generates a perceived sensorial indicia (sound generation) of discrete event occurrence and further includes a perceived sensorial reinforcer (color generation) which increases probability that the composition will be used until discrete event occurrence.

FIG. 5 provides a graph which shows rate of release of materials from capsules in accordance with the invention to generate a perceived sensorial indicia (generation of color 3) of discrete event occurrence and further includes a plurality of perceived sensorial reinforcers (generation of color 1 and generation of color 2) to increase probability that the composition will be used until the discrete event occurrence.

FIG. 6 provides a graph which shows rate of release of materials from capsules in accordance with the invention to generate perceivable sensorial indicia of achieving particular application characteristics (an amount or level of force applied between surfaces to which the cosmetic is applied) and further provides a second perceivable sensorial indicia as to when the cosmetic becomes efficacious.

### V. MODE(S) FOR CARRYING OUT THE INVENTION

Compositions which provide encapsulated material released to generate perceivable sensorial attributes or perceived sensorial indicia of discrete event occurrence and can further provide perceived sensorial reinforcers to increase or decrease probability of composition use coincidence with occurrence of such discrete event.

Compositions in accordance with the invention means formulations useful with respect to numerous and varied applications including but not limited to cleaning formulations; pharmaceutical formulations; surface preparation or finishing formulations such as automotive finish cleaners and waxes; upholstery cleaners; carpet cleaners; or the like which provide a carrier(s) to conveys capsules containing materials which are released to provide, individually or in combination, perceivable sensorial attributes, perceivable sensorial indicia of discrete event occurrence, or perceivable sensorial reinforcers; and non-aqueous carrier(s)(miscible in water) to convey capsules and materials soluble, activated, or miscible in water, aqueous, aqueous solutions, or other aqueous containing materials; and non-aqueous carrier(s)(miscible in water) to convey aqueous stable capsules containing materials soluble, activated, or miscible in water, aqueous, aqueous solutions, or other aqueous containing materials, whether or providing individually or in combination, perceivable sensorial attributes, perceivable sensorial indicia of discrete event occurrence, or perceivable sensorial reinforcers.

Cosmetic(s) means, individually or in combination, elements, substances, compositions, components, or materials, that are suitable for application to the human skin, hair, or nails such as soaps, shampoos, conditioners, moisturizers, masks, depilatories, lotions, creams, toothpastes, teeth whiteners, make up removers, or the like.

Carriers in accordance with the invention means any non-aqueous but water miscible carrier or aqueous carrier, or combination of such carriers, in which capsules containing water miscible or activated materials can be maintained without substantial degradation and conveyed to the application location for use including, but not limited to, those carriers described in the examples herein, or one or more of: ethylene glycol, polyethylene glycol, polypropylene glycol, 1,4-butanediol, 1,5-pentanediol, 1,6-hexanediol, pentaelytluitol, neopentyl glycol, glycerol, sorbitol, erithrithol, methanol, ethanol, isoproanol, 1-propanol, pentanol, hexanol, phenoxyethanol, benzyl alcohol, or the like, individually or in various combinations. This list is not meant to be exhaustive and is intended to provide a sufficient number of examples of the wide variety of carriers in accordance with the invention so that one of ordinary, skill in the art can make and use the invention, whether the embodiments are utilized for cosmetics, or otherwise.

Compositions or cosmetics in accordance with the invention can further include a surfactant for the purpose of forming homogeneous solutions, or for dispersion or suspension of components for the purpose of creating a heterogeneous system such as an emulsion or suspension. The surfactants can be non-aqueous but water miscible for the purpose of cleaning and being free-rinsing. Surfactants may be generically selected from the following classes: anionic, cationic, nonionic, and amphoteric.

Anionic surfactants include for example sodium toluene sulfonate, sodium naphthalene sulfonate, sodium lauryl sulfate, or the like. Cationic surfactants include for example alkyl, aryl or alkyl aryl amines, or the like. Nonionic surfactants include for example alkyl aryl ethoxylates, alkyl ethoxylates, alkyl aryl propoxylates and alkyl propoxylates. Amphoteric surfactants are those having a positive and negative charge within the same molecule and are therefore a blend of cationic and anionic. A common example is the class of betaine surfactants. These lists are intended to examples of surfactants that can be used in accordance with the invention and provide a sufficient number of examples of the wide variety of surfactants that can be used according to the invention so that one of ordinary skill in the art can make and use the invention.

Hydrophile/lipophile balance (HLB) can be important with respect to certain cleaning composition embodiments of the invention. Surfactants at 10 are exactly balanced between hydrophilic and lipophilic. Less than 10 becomes more lipophilic (also synonymously oleophilic). Greater than 10 becomes increasingly hydrophilic. For certain embodiments of cleaning agents encompassed by the invention the range can be between about 10 to about 40. A more preferred range as to some cleaning agent embodiments of the invention may be between about 12 to about 30 and as to certain embodiments of the invention the most preferred range may be between about 15 to about 25.

Capsules in accordance with the invention are combined, mixed, or blended with the carrier and provide a central reservoir in which one or more materials, as described below, are contained or sequestered by one or more capsule shell(s), capsule wall (s), or capsule coats coascervated, formed, or applied by fluid bed coating (spraying of a liquid on fluidized powder or particles to uniformly coat the particles) of urea-formaldehyde, various forms of gelatin, polyvinyl alcohol, polyvinyl acetate, fully hydrolyzed polyvinyl alchohol, Celvol 107, vinylidene chloride-methyl acrylate copolymer, Daran 159 Latex, polyvinyl pyrrolidone, polyvinyl methyl ether, polyvinyl methyl ether/maleic anyhydride colpolymer, carboxy methyl cellulose, hydroxy ethyl cellulose, cellulose acetate butyrate, cellulose acetate propionate, polyvinyl alcohol/acetyl copolymer, or as described by U.S. Pat. Nos. 2,800,457; 3,697,437; 3,336,155, to prevent or avoid release, combination or reaction, or entrainment of such materials with the carrier.

As such, the composition of the carrier and the composition of the capsules are selected to provide capsules which are stable or inert within the carrier but either solublize in response to application conditions to release materials, or are ruptured in response to application force characteristics, or the capsules are soluble in application conditions allowing rupture by application force characteristics. Capsules are sufficiently stable or inert, if the capsule when entrained in the carrier substantially prevents the material within from being transferred to the carrier, during the period prior to use. As to certain embodiments of the invention, the capsule may have to be stable in the carrier for only a few minutes while as to other embodiments of the invention the capsule may have to be stable to the carrier for a year, or longer.

Application force characteristics can vary with respect to the applications in which compositions in accordance with the invention are utilized. Application force characterics include the pressure, shear, or other forces generated using the composition for the intended application. For example, and washing agents in accordance with the invention are introduced to application force characteristics generated by hand washing and comprise the pressure, shear, or forces typically generated by rubbing the composition over or between the surfaces of the hands.

Application conditions can also vary with respect to the applications in which compositions in accordance with the invention are utilized. Certain application conditions may expose the composition to water or other aqueous solutions or substances, while other application conditions may utilize the composition without substantial exposure to any other substance, or may expose the composition to non-aqueous solutions or substances.

Material contained within the capsules may be aqueous soluble material or non-aqueous soluble material, including, but not limited to: surfactants, enzymes, flavors, fragrances, bleach or bleaching agents, pH change indicators, dyes, color, antistatics, fabric softener, lubricants, emollients, insecticides, disinfectants, perfume, dentifrice, vaccines, drugs, medications, amino acids, nucleic acids, microbes, hormones, antiviral proteins, antiviral peptides, industrial chemicals (which includes a wide variety of materials such as oxidizing agents, reducing agents, free radical initiators, or the like), bioactive agents, lotions, gels, or the like, individually or in combination, to impart upon release from the capsule: emolliency, moisturizing, lubricity, color, fragrance, texture, viscosity, sound, or other perceivable sensorial attributes.

As to certain embodiments of the invention, the composition, configuration, or number of the capsules, or combination of different types of capsules (whether aqueous soluble or insoluble), are selected so that release of material provides notice of occurrence of a discrete event (which may or may not be otherwise perceivable to the user). Generation of such perceivable sensorial indicia in different application conditions or in response to different application force characteristics necessitates adjusting capsule rupture charateristics to coordinate release of material from the capsule to generate indicia sufficiently coincident with discrete event occurrence to allow action in response to the occurrence of the discrete event, whether the discrete event is perceivable or not.

A non-limiting example of a liquid hand washing agent in accordance with the invention provides a carrier which can be used for hand washing entraining one or more populations of carrier stable capsules which contain material subsequently released during the hand washing event to generate at least one perceivable sensorial indicia that sufficient hand washing has been achieved. Alternate embodiments of the hand washing agent in accordance with the invention can coordinate generation of the perceivable sensorial indicia upon achieving pre-determined hand washing force characteristics, or alternately elapse of a duration of hand washing time.

A specific non-limiting example of an embodiment of the hand washing agent invention can be used by applying an amount of the hand washing agent to the surface of the hands, rubbing the hand washing agent on or between the surfaces of the hands in combination with water for about 12 seconds to about 15 seconds, perceiving color change which indicates hand washing has been completed, rinsing the hand washing agent from the surface of the hands.

With respect to cleaning agents, including but not limited to hand washing agents or composition, which are alkaline, placing the carbonate in the carrier and the acidic material in a capsule may be most practical. Therefore, sodium carbonate or sodium bicarbonate as examples could be added to the carrier composition. An acidic material such as sodium acetate can be encapsulated. When the capsule releases the sodium acetate through rupture or dissolution, the immediate environment experiences a drop in pH. This relative acidic environment destabilizes the carbonate resulting in the release of carbon dioxide. The immediate effect would be foam expansion and sound created through the breaking of the bubbles. The sound measured in decibels can be in the range of 0.5-12 db generated through the rapid breaking of a myriad of CO₂ bubbles. Carbonates used may be the lithium, sodium, potassium or ammonium carbonates or the bicarbonates (aka monohydrogen carbonate). Carbonates and bicarbonates of di- and tri-valiant alkali metal salts such as calcium, barium, iron, aluminum, or the like, may be less preferred due to inherently poor solubility. Acids and acidic materials more preferred may be the salts of strong mineral or organic acids or the weak organic acid. Free acids would be characterized by citric acid, ascorbic acid, tartaric acid, and the like. Salts of acids include zinc chloride, sodium sulfate, monosodium phosphate, sodium acetate, or the like.

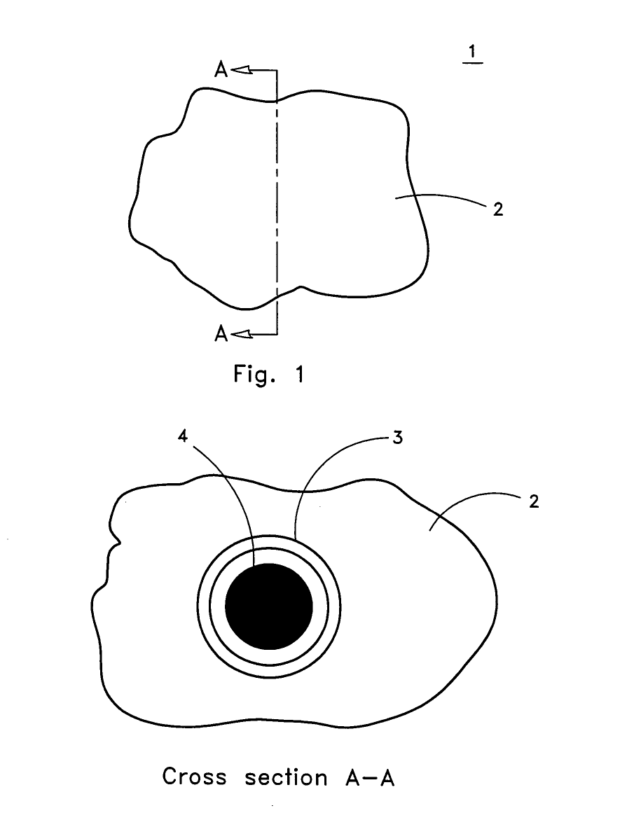

Now referring primarily to FIG. 1, a composition (1) in accordance with invention can comprise a carrier (2) entraining capsules (3) containing at least one material (4) released in response to pre-determined application force characteristics, pre-determined application conditions, or elapse of time to generate at least one perceived sensorial attribute, at least one perceived sensorial indica of discrete event occurrence, or at least one perceived sensorial reinforcer, individually or in combination.

Now referring primarily to FIGS. 1 through 3, basic embodiments of the invention can provide a cosmetic, or method of cosmetic use, which in whole or in part, achieves desired efficacy by application force(s) generated by contact between anatomical surfaces and the cosmetic during use. These embodiments of the cosmetic can provide capsules stable in the carrier and stable to application conditions and which rupture to release material in response to generation of pre-determined application force characteristics to generate perceived sensorial indicia that the cosmetic has been used properly.

Alternately, basic embodiments of the invention can provide a cosmetic, or a method of cosmetic use, which in whole or in part, achieves desired efficacy by elapse of a duration of time during use. These embodiments of the invention can provide capsules stable in the carrier and which are solubilized in application conditions to the extent necessary to rupture in response to application force characteristics after elapse of a pre-determined time of use.

The range of time for release of materials in the hand washing agents described by the examples below can be from substantially instantaneous up to about 150 seconds. Release of materials can occur in a narrow time frame with capsules of uniform kind and type, or release of materials can occur as a gradient through the use of mixture of capsule types or kinds. Material contained by a population of capsules of uniform configuration can be released in a window of about t+/−2 seconds, or alternatively mixed populations of capsules can be established to allow staged release of the contents starting at a specified time and continuing incrementally over any specified time duration up to about 150 seconds, or beyond.

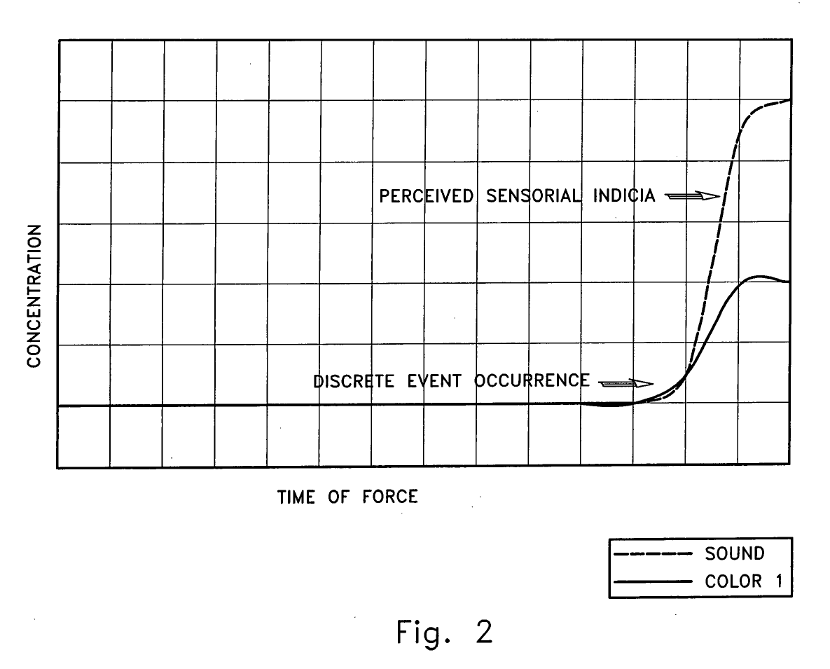

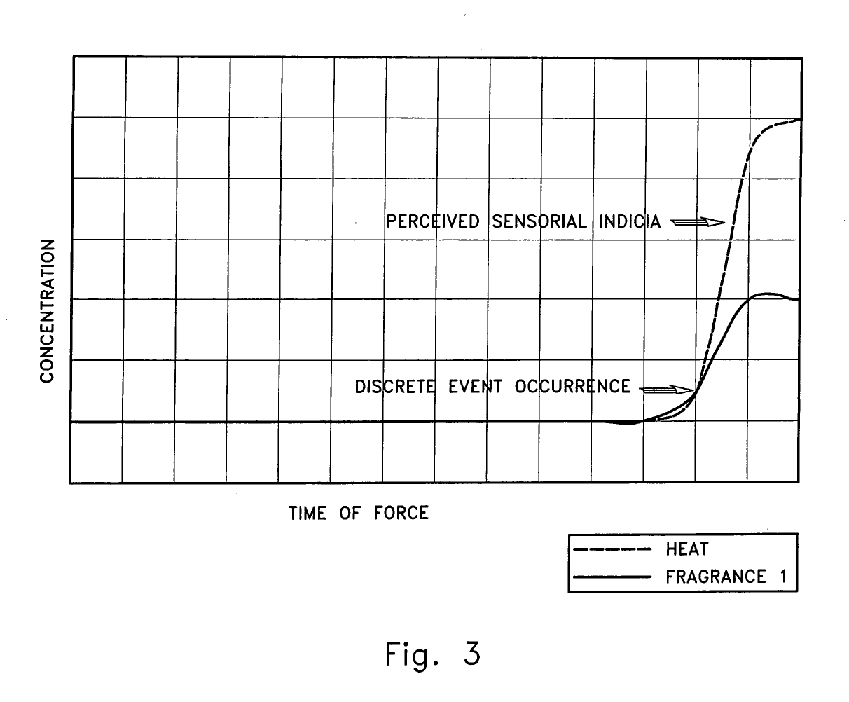

Again referring primarily to FIGS. 2 and 3, perceived sensorial indicia of discrete event occurrence can be generated by release of a colored material, a scented material, or the like, or generated by release of a material which reacts with a component in the carrier to generate change, color change, fragrance, heat, or sound, among others. Importantly, the entrainment of capsules in the carrier of the cosmetic provides the user, upon rupture of the capsule wall, with more than a perceived sensorial attribute, but rather a perceived sensorial indicia of a discrete event occurrence informing the user concerning the status of the cosmetic, the efficacy of its use, or other information on which the user can act.

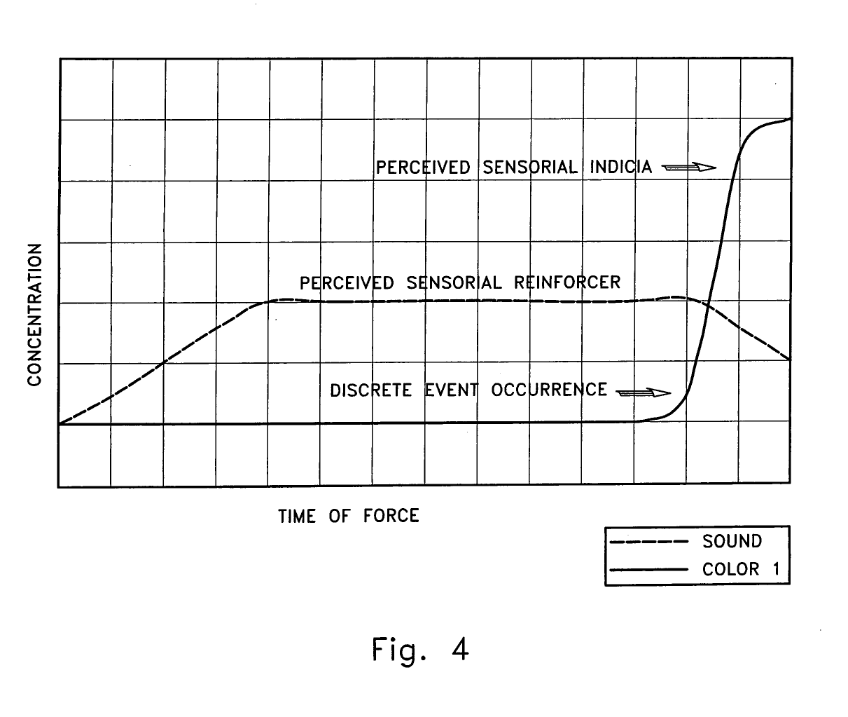

Now referring primarily to FIG. 4, cosmetics in accordance with the invention can further provide perceivable sensorial reinforcers. Some of the perceivable sensorial reinforcers can be utilized in accordance with operant conditioning theory to increase, or in some instances decrease, the probability that the cosmetic agent will be in use when a discrete event occurs or when the perceivable sensorial indicia of discrete event occurrence as described above is generated. As such, in conjunction with or separate from the use of a perceived sensorial indicia of discrete event occurrence the invention can further include the release of the same or different materials from the same or different capsules used to generate perceivable sensorial reinforcers, as described above. The profile of release rate for materials used as perceivable sensorial reinforcers can be tailored based upon field trials of sample products.

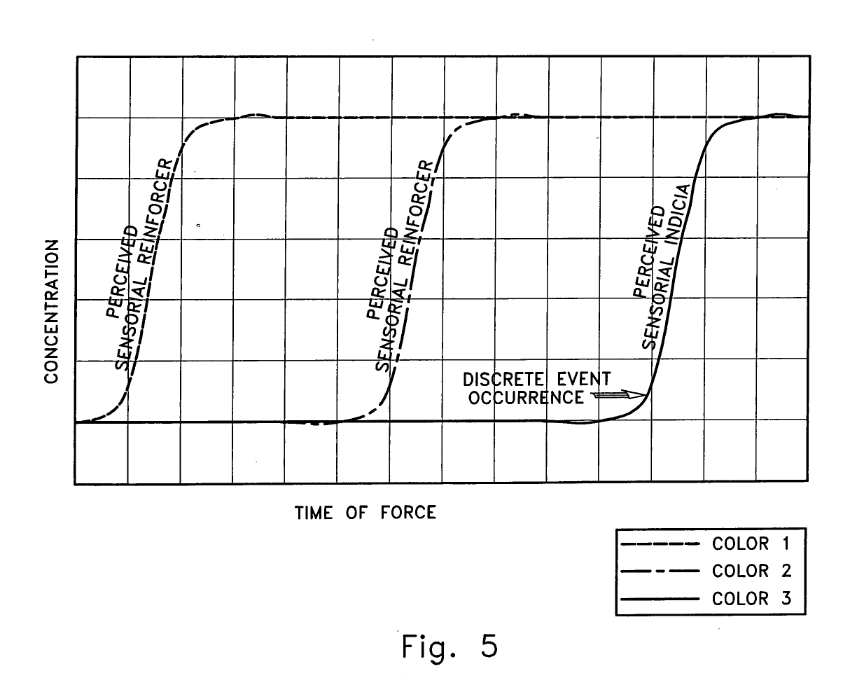

Now referring primarily to FIG. 5, certain embodiments of the invention, provide staged release of perceivable sensorial reinforcers, perceivable sensorial attributes, and perceivable sensorial indicia. Capsules of various types or kinds can be mixed to generate simultaneous or serial generation of perceivable sensorial attributes, perceivable sensorial indica, or perceivable sensorial reinforcers.

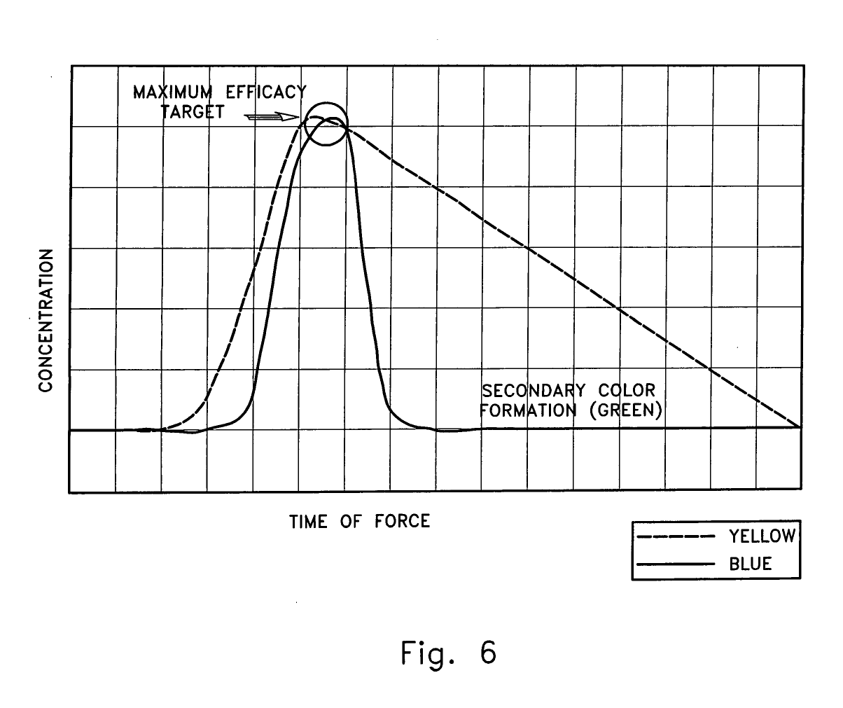

Now referring primarily to FIG. 6, hand washing with a hand washing agent in accordance with the invention can provide capsules which rupture in response to hand washing application force characteristics to release a blue colored material (perceivable sensorial reinforcer), and further provide aqueous soluble capsules which rupture in response to solubilization in water to release a yellow colored material. An amount of the hand washing agent is applied to the hands and upon application of pre-determined hand washing force characteristics blue colored material is released. Then upon elapse of a pre-determined duration of time the yellow colored material is released. Only upon the coincidence of the desired application of force with the elapse of the desired time will the cosmetic, or foam generated by hand washing, turn green providing the perceived sensorial indicia of desired product efficacy or desired manner of product use.

Serial release of materials to generate perceived sensorial indicia of product efficacy for the hand washing agent described above can also be realized through the encapsulation of capsules. A first material can be contained in a first capsule to provide a perceived sensorial reinforcer. A second material can be coated on the first capsule surface and the entirety subsequently contained within a second capsule to provide a second perceived sensorial reinforcer. The process can be repeated a 2 or 3 times, or more, as may be desired. The release of the first material can provide a perceivable sensorial reinforcer (generation of yellow color) and the release of the second material in combination with the first can provide a perceivable sensorial indica when coordinated with a discrete event.

With respect to one of the various embodiments of the invention, application force characteristics necessary to rupture the outside capsule may be less than that for the encapsulated capsule. As such, rupture of the outside capsule could release for example a blue dye, while the inner capsule soluble in the application conditions can for example release yellow dye providing the perceived sensorial indicia of green color change after the colored materials mix. In this case, the desired application of force would be a prerequisite to the timing event provided by the encapsulated aqueous soluble capsule. As such, a level of hand washing forces are first achieved prior to initiating timing of the actual washing period. Alternately, the soluble capsule could be located outside while the friable capsule is located inside. In that case, a desired amount of time can elapse using the product before application of the desired force would then generate the perceived sensorial indicia of green color indicating that sufficient hand washing has been achieved.

FIGS. 1 to 6, along with the description and non-limiting examples below illustrate how numerous embodiments of the cosmetic invention can be made and used to provide perceived sensorial indicia of discrete event occurrence and how perceivable sensorial reinforcers are utilized in relation to such a discrete event occurrence to increase the probability of the cosmetic being used until the occurrence of the discrete event.

Understandably, there are numerous combinations and permutations of perceivable sensorial indicia that can be used individually or in combination with perceivable sensorial reinforcers as described above, provided by the non-limiting examples, or as shown in the Figures. This disclosure is not intended to be limiting with respect to the type or kind of cosmetic; the type or kind of use the cosmetic may be applied to; the type, kind, or duration of discrete event occurrence; the duration of time from the initial application of the cosmetic to the coincidence of the discrete event occurrence with the perceived sensorial indicia; the duration or concentration of the perceived sensorial indicia; the number, type or kind of perceived sensorial indicia contained or conveyed by the cosmetic; the concentration of the perceived sensorial indicia; the manner of temporal coupling of the perceived sensorial indicia to the discrete event occurrence so long as a relationship between the perceived sensorial indicia and the occurrence of the discrete event can be useful in achieving the intended consequence or proper use of the cosmetic; the number type or kind of perceived sensorial reinforcer; the concentration or duration of the perceived sensorial reinforcer so long as the perceived sensorial reinforcer increases the probability or decreases the probability of coincidence of cosmetic use with the occurrence of the discrete event.

The following examples are not intended to limit the scope of the invention solely to the compositions described, but in conjunction with the description above, are intended to provide a sufficient number of examples so that a person of ordinary skill in the art would know how to make and use the wide variety of cosmetics in accordance with the invention including the numerous embodiments of the invention (hand washing agents or otherwise) which provide a non-aqueous carrier in which aqueous activatable components are added, or those numerous embodiments of the invention that provide perceivable sensorial indicia, or those numerous embodiments of the invention that provide perceivable sensorial reinforcement attributes, individually or in combinations thereof.

### EXAMPLE 1

A composition to treat the skin in general and particularly the skin of the hands, can be prepared as follows:

A carrier can be produced by combination of 125 parts of water, 18 parts of sodium xylene sulfonate, 5 parts of sodium toluene sulfonate, 1 part of sodium dodecylbenzene sulfonate, and 10 parts of dodecyl phenol polyoxyethylene ethanol. The combination can be thickened by adding 6 parts of an aqueous solution of polyacrylamide (12% wt/wt) and brought to a neutral pH using a 50% solution of sodium hydroxide.

An capsule containing dye or other colored material can be produced by providing a seed particle of sugar sized to about 100 microns which can be coated with blue dye #7 to generate a blue dye #7 sugar particle having a diameter of about 150 microns. The blue dye #7 sugar particle can be encapsulated with polyvinyl acetate using the process of fluid bed coating or Wuerster process to a final nominal diameter of about 200 microns. Fluid bed coating of the blue dye #7 sugar particle provides a capsule having a wall thickness of about 50 microns.

The capsules can be combined with the above-described carrier to provide a hand washing agent. The hand washing agent can be applied to the hands, hand washing commences, and hand washing application forces are applied to the capsules containing the blue dye #7 sugar particle. The capsule wall configured as above-described ruptures or otherwise releases the blue dye #7 sugar particle in response to such hand washing application forces. The released blue dye #7 combines with the foam generated by the carrier to change the foam to a blue color. The release of the blue dye #7 is delayed from the commencement of hand washing to provide a change in foam color from white to blue in about 15 seconds. The change in foam color provides a perceivable sensorial indicia that ample time has elapsed for the hand washing event using the above-described carrier.

### EXAMPLE 2

A carrier was prepared as described in Example 1 through the blending of surfactants and thickening with polyacrylamide.

Capsules containing oils having various fragrances were separately generated using the process of coacervation wherein dropelets of an oily substance are dispersed in water, coated with gelatin, and hardened with gluteraldehyde. Methyl salicylate or oil of wintergreen can be encapsulated in this manner. Six parts of the capsules produced as above-described containing methyl salicylate or oil of wintergreen-were added to the carrier to produce a hand washing agent.

Conditions of coacervation were then adjusted by trials to generate a capsule having a wall configured to rupture, fracture, or otherwise release oils to generate a perceivable sensorial indicia corresponding to effective hand washing with the above-described carrier (about ten seconds after commencement of hand washing) to an otherwise substantially odorless hand washing event.

### EXAMPLE 3

A hand washing agent was prepared by adding the following ingredients:

A carrier was prepared using about 90 parts of water, about 8 parts of sodium lauryl sulfate, about 4 parts of isooctylphenoxypolyoxyethylene ethanol (12 moles EO) and about 1.5 parts of sodium carbonate. When blended, the solution was thickened by using 4 parts of a medium molecular weight methyl cellulose.

Neat sodium acetate particles sized to about 125 microns were coated with polyvinyl acetate using the fluid bed coating process to provide a capsule of sodium acetate having a nominal diameter of 170 microns. Seven parts of the sodium acetate capsules were combined with the carrier with no apparent visible change to the carrier.

An amount of the hand washing agent applied to the hands followed by hand washing ruptured or fractured capsules to release sodium acetate into the carrier. The acidic sodium acetate reacts with the alkaline sodium carbonate in the carrier to generate carbon dioxide bubbles in the carrier sufficient that bubbles breaking produce an audible sound about ten seconds from commencement of the hand washing event.

### EXAMPLE 4

A plurality of perceivable sensorial indicia can be generated and controlled as independent functions, singularly or collectively, to combine the effects of color, sound, and fragrance in various combinations and permutations.

To the carrier described in Example 3, 4 parts of a blue dye #7, 5 parts of methyl salicylate, and 7 parts of sodium acetate, all of which were encapsulated using the processes above-described were added.

An amount of the composition was poured on the hands, hand washing commenced, and an adequate percentage of the capsules were ruptured so as to allow a color change of the carrier foam from white foam to blue foam about 15 seconds after commencement of hand washing. Wintergreen fragrance was generated from the heretofore non-scented carrier, an audible sound was generated, and altered tacticity due to the expansion and effervescence of the carrier was percieved.

### EXAMPLE 5

A composition for cleaning skin was prepared as follows:

A non-aqueous carrier was prepared by combining 100 parts of polyethylene glycol, 15 parts of tridecyl polyoxyethylene ethanol (15 moles EO), 5 parts of nonyl phenol polyoxyethylene ethanol (18 moles EO), and 0.06 parts of a 1% (w/v) solution of phenolphthalein in ethanol.

To the above composition were added 6 parts of encapsulated trisodium phosphate (TSP) prepared by fluid bed coating of TSP beads having a nominal size of about 75 microns, although the particle size distribution ranged from about 40 microns to about 180 microns, with as a 4% solution out of water/methanol solution (60:40) fully hydrolyzed polyvinyl alcohol (such as Celvol 107 as supplied by Celanese) such that the nominal diameter increases to about 120 microns. The capsules sized after coating generate two lots; a first lot of greater than about 100 microns and a second lot of less than about 100 microns.

The composition was applied to the hands followed by about an equal amount of water, hand washing commenced, and there was a change in the color of the composition from clear to a red color. This is due to the release of TSP after solubilization and rupture of the polyvinyl alcohol capsule wall, which elevates the carrier pH from acidic to slightly alkaline altering perceived color of the TSP.

Time control of color change can be controlled by altering the size of the capsule. Capsules of less than about 100 microns generate color change of the carrier after elapse of about 30 seconds of hand washing. Capsules of greater than about 100 microns generate color change of the carrier after elapse of about 5 to about 8 seconds of hand washing. Distribution of capsule size in the carrier can be adjusted to generate color change in the carrier after elapse of between about 5 and about 30 seconds of hand washing.

### EXAMPLE 6

A composition for cleaning skin was prepared as follows:

A non-aqueous carrier was prepared by combining 150 parts of glycerin, 18 parts of tridecyl polyoxyethylene ethanol (15 moles EO), 10 parts of dodecyl phenol polyoxyethylene ethanol (22 moles EO), and 0.08 parts of a 1% (w/v) solution of phenolphthalein in ethanol.

To the above composition were added 9 parts of encapsulated trisodium phosphate (TSP) prepared by fluid bed coating of TSP beads having a nominal size of about 75 microns, although the particle size distribution ranged from about 40 microns to about 180 microns, with a 25% solution out of water as an emulsion vinylidene chloride-methyl acrylate copolymer (Daran 159 Latex) such that the nominal diameter increases to about 140 microns. The capsules sized after coating generate two lots; a first lot of greater than about 100 microns and a second lot of less than about 100 microns.

The composition was applied to the hands followed by about an equal amount of water, hand washing commenced, and there was a change in the color of the composition from clear to a red color as above-described due to the release of TSP after solubilization and rupture of the capsule wall, which elevates the carrier pH from acidic to slightly alkaline altering perceived color of the TSP.

Time control of color change can be controlled by altering the size of the capsule. Capsules of less than about 100 microns generate color change of the carrier after elapse of about 45 seconds of hand washing. Capsules of greater than about 100 microns generate color change of the carrier almost immediately after commencement of hand washing. Distribution of capsule size in the carrier can be adjusted to generate color change in the carrier almost immediately after hand washing commences to about 45 seconds after hand washing commences.

As can be easily understood from the foregoing, the basic concepts of the present invention may be embodied in a variety of ways. The invention involves numerous and varied compositions and methods of making and using such compositions; products in or with which such compositions can be used; and devices which can be utilized to make the compositions or products, facilitate the methods of using the compositions, or used with the compositions, in various combinations or permutations.

As such, the particular embodiments or elements of the invention disclosed by the description or shown in the figures accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus disclosed may be disclosed as the physical element or the action which that physical element facilitates. As but one example, the disclosure of a “capsule” should be understood to encompass disclosure of the act of “encapsulating”—whether explicitly discussed or not—and, conversely, were there effectively disclosure of the act of “encapsulating”, such a disclosure should be understood to encompass disclosure of a “encapsulated” and even a “means for encapsulating.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

Thus, the applicant(s) should be understood to claim at least: i) each of the compositions, cosmetic products, and devices as herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The claims set forth in this specification are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

The claims set forth below are intended describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A composition, comprising:
a. an aqueous carrier;
b. a plurality of capsules entrained in said aqueous carrier, wherein each of said capsules contain an amount of material, and wherein said each of said capsules has a capsule wall with at least one adjustable capsule rupture characteristic to vary delay of release of said material; and
c. a perceivable sensorial indicia generated by release of said material from said plurality of capsules coordinated with occurrence of a discrete event.

2. A composition as described in claim 1, wherein said plurality of capsules comprise a plurality of non-aqueous soluble capsules.

3-31. (canceled)

32. A composition as described in claim 2, wherein said at least one adjustable capsule rupture characteristic comprises a capsule wall thickness.

33. A composition as described in claim 32, wherein said at least one adjustable capsule rupture characteristic comprises a capsule size.

34-36. (canceled)

37. A composition as described in claim 33, wherein said capsule wall thickness is between about 10 microns and about 30 microns.

38. A composition as described in claim 37, wherein said capsule size is between about 60 microns and about 240 microns.

39. A composition as described in claims 38, wherein said composition comprises a hand washing agent.

40-44. (canceled)

45. A composition as described in claim 39, wherein said at least one capsule rupture characteristic of said plurality of capsules adjusts to release said material between about 5 seconds and about 30 seconds after commencement of a hand washing event.

46. A composition as described in claim 39, wherein said at least one capsule rupture characteristic of said plurality of capsules adjusts to release said material between about 5 seconds and about 15 seconds after commencement of a hand washing event.

47. A composition as described in claim 45, wherein said capsules are greater than about 100 microns in size.

48. A composition as described in claim 46, wherein said capsules are less than about 100 microns in size.

49. A composition as described in claim 46, wherein said perceivable sensorial indicia generated by release of said material from said plurality of capsules comprises color change of said aqueous carrier.

50. A composition as described in claim 49, wherein said discrete event comprises achievement of a therapeutic hand wash event.

51. A composition as described in claim 50, wherein said discrete event comprises elapse of a hand wash event of pre-determined duration.

52. A composition as described in claim 51, wherein said discrete event comprises elapse of a hand wash event having duration of time selected from the group of: between about 5 seconds and about 10 seconds, between about 6 seconds and 11 seconds, between about 7 seconds and about 12 seconds, between about 8 seconds and about 13 seconds, between about 10 seconds and about 14 seconds, between about 111 seconds and about 15 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds.

53-57. (canceled)

58. A composition as described in claims 52, wherein said aqueous carrier further comprises an amount of color change material responsive to pH change of said aqueous carrier.

59. A composition as described in claims 58, wherein said material within said plurality of capsules comprises an aqueous carrier pH alteration material.

60. A composition as described in claim 59, wherein said an amount of color change material responsive to pH change of said aqueous carrier comprises phenolphtalein.

61. A composition as described in claim 60, wherein said carrier comprises a mixture of polyethylene glycol, tridecyl polyoxyethylene ethanol, nonyl phenol polyoxyethylene ethanol, and phenolphthalein.

62. A composition as described in claim 61, wherein said carrier comprises a mixture of about 100 parts polyethylene glycol, about 15 parts tridecyl polyoxyethylene ethanol, about 5 parts nonyl phenol polyoxyethylene ethanol, and about 0.06 parts of a 1% (w/v) solution of phenolphthalein.

63. A composition as described in claim 62, wherein said carrier comprises a mixture of glycerin, tridecyl polyoxyethylene ethanol, dodecy phenol polyoxyethylene ethanol, and phenolphthlein.

64. A composition as described in claim 63, wherein said carrier comprises a mixture of about 150 parts glycerin, about 18 parts tridecyl polyoxyethylene ethanol, about 10 parts dodecy phenol polyoxyethylene ethanol, and about 0.08 parts of a 1% (w/v) solution of phenolphthlein.

65. A composition as described in claim 64, wherein said amount of material contained by said plurality of capsules comprises trisodium phosphate.

66. A composition as described in claim 65, wherein said capsules are formed from a capsule material selected from the group consisting of a urea-formaldehyde, a polyvinyl acetate, a vinylidene chloride-methyl acrylate copolymer, a Daran 159 Latex, a polyvinyl methyl ether/maleic anyhydride colpolymer, a cellulose acetate butyrate, Celvol 107, a fully hydrolyzed polyvinyl alcohol, and a cellulose acetate propionate.
